(19) United States
(12) Patent Application Publication
(10) Pub. No.: US 2025/0228815 A1
(43) Pub. Date: Jul. 17, 2025

(54) LYOPHILIZED RESINIFERATOXIN

(71) Applicant: Gruenenthal GmbH, Aachen (DE)

(72) Inventors: Zouaoui BOUREZG, Aachen (DE); Susanne FORNERO QUAACK, Aachen (DE)

(73) Assignee: Gruenenthal GmbH, Aachen (DE)

(21) Appl. No.: 19/021,874

(22) Filed: Jan. 15, 2025

(30) Foreign Application Priority Data
Jan. 15, 2024 (EP) 24151807.5
Jan. 22, 2024 (EP) 24153125.0
Jan. 22, 2024 (EP) 24153126.8
May 13, 2024 (EP) 24175474.6

(51) Int. Cl. A61K 31/357; A61K 9/00; A61K 9/19; A61K 47/10; A61P 29/02

(52) U.S. Cl. CPC A61K 31/357; A61K 9/0019; A61K 9/19; A61K 47/10; A61P 29/02

(57) ABSTRACT

The invention relates to a reconstitutable solid pharmaceutical composition comprising or essentially consisting of Resiniferatoxin in an amount of less than 3.20 μg, preferably at most 900 ng, more preferably at most 500 ng; optionally, a surfactant; and optionally, a buffer. The composition according to the invention is particularly useful for the treatment of pain due to knee osteoarthritis. Prior to administration, the solid composition is reconstituted with a reconstitution liquid thereby obtaining a reconstituted composition. Said reconstituted composition or a partial volume thereof is subsequently administered as dose unit, preferably by intraarticular injection into the knee.

Priority is claimed of European Patent Application No. 24 151 807.5, filed on Jan. 15, 2024; European Patent Application No. 24 153 125.0, filed on Jan. 22, 2024; European Patent Application No. 24 153 126.8, filed on Jan. 22, 2024; and European Patent Application No. 24 175 474.6, filed on May 13, 2024, the disclosures of which patent applications are incorporated herein by reference.

The invention relates to a reconstitutable solid pharmaceutical composition comprising or essentially consisting of Resiniferatoxin in an amount of less than 3.20 μg, preferably at most 900 ng, more preferably at most 500 ng; optionally, a surfactant; and optionally, a buffer. The composition according to the invention is particularly useful for the treatment of pain due to knee osteoarthritis. Prior to administration, the solid composition is reconstituted with a reconstitution liquid thereby obtaining a reconstituted composition. Said reconstituted composition or a partial volume thereof is subsequently administered as dose unit, preferably by intraarticular injection into the knee.

Resiniferatoxin (RTX) is a naturally occurring compound produced by the Moroccan cactus like succulent Euphorbia resinifera. Resiniferatoxin is known as a transient receptor potential cation channel subfamily V member 1 (TRPV1) agonist. RTX is an ultrapotent capsaicin analog with a unique spectrum of pharmacological actions. The therapeutic window of RTX is broad, allowing for the full desensitization of pain perception and neurogenic inflammation without causing unacceptable side effects. Intravesical RTX was shown to restore continence in a subset of patients with idiopathic and neurogenic detrusor overactivity. RTX can also ablate sensory neurons as a “molecular scalpel” to achieve permanent analgesia. This targeted (intrathecal or epidural) RTX therapy holds great promise in cancer pain management. Intraarticular RTX is undergoing clinical trials to treat moderate-to-severe knee pain in patients with osteoarthritis. Similar targeted approaches may be useful in the management of post-operative pain or pain associated with severe burn injuries (P. Campiglia et al., Int J Mol Sci 2023, 24(20), 15042).

Resiniferatoxin has been suggested for treating various types of pain and various other disorders and conditions by various routes of administration.

C. Sila et al., BJU Int. 2001 September;88(4):355-60. doi: 10.1046/j.1464-410x.2001.02339.x. report that seven patients with detrusor hyperreflexia were treated intravesically with Resiniferatoxin dissolved in 10% ethanol in saline (50 nmol/L solution in two and 100 nmol/L in five).

H. Kuo, J Urol. 2003 September;170(3):835-9. doi: 10.1097/01.ju.0000081652.31524.27 discloses intravesical Resiniferatoxin therapy with 10 ml of 100 nM Resiniferatoxin in 10% ethanol solution for 40 minutes.

M. de Séze et al., J Urol. 2004 January;171(1):251-5. doi: 10.1097/01.ju.0000100385.93801.d4 relates to a comparison of the efficacy and tolerability of nonalcohol capsaicin vs. Resiniferatoxin in 10% alcohol in neurogenic patients with detrusor hyperreflexia.

C. Silva et al., Eur Urol. 2005 October;48(4):650-5. doi: 10.1016/j.eururo.2005.04.012 relates to an investigation wherein patients with spinal neurogenic detrusor overactivity were randomized to receive intravesically 50 nM Resiniferatoxin dissolved in 10% ethanol in saline.

E. Y. Kissin et al., Anesth Analg. 2005 November; 101(5):1433-1439. doi: 10.1213/01.ANE. 0000180998.29890.B0. relates to the effects of intraarticular Resiniferatoxin in experimental knee-joint arthritis.

J. C. Shin et al., Spinal Cord. 2006 May; 44(5):309-14. doi: 10.1038/sj.sc.3101851 relates to an investigation wherein 100 ml of Resiniferatoxin solution, at a concentration of 100 nM diluted in 10% ethanol, was intravesically instilled into the bladder of 15 spinal cord injury patients with neurogenic detrusor overactivity.

C. Silva et al., BMC Urol. 2007 Jun. 11:7:9. doi: 10.1186/1471-2490-7-9 relates to an investigation wherein patients with overactive bladder were instilled with 100 ml of 50 nM Resiniferatoxin in 10% ethanol in saline.

I. Vetter et al., Eur J Pain. 2008 May; 12(4):441-54. doi: 10.1016/j.ejpain.2007.07.001 report that ethanol can sensitize TRPV1-mediated responses, but the pathways contributing to the potentiation of TRPV1 by ethanol have not been clearly defined. The presented results suggest that morphine may be of limited use in inhibiting nociceptive TRPV1 responses that have been sensitized by exposure to ethanol.

I. Kissin et al., Curr Top Med Chem. 2011; 11(17):2159-70. doi: 10.2174/156802611796904924 reviews therapeutic targeting of TRPV1 by Resiniferatoxin, from preclinical studies to clinical trials.

M. J. Iadarola et al., Resiniferatoxin for Pain Treatment: An Interventional Approach to Personalized Pain Medicine, Open Pain J. 2013; 6: 95-107 reviews preclinical and clinical studies related to Resiniferatoxin. Pain conditions that may be susceptible to treatment with local injection or topical Resiniferatoxin include osteoarthritis, whereas Resiniferatoxin is directly injected into the joint. No dosages are mentioned.

Y. Kim et al., The effects of intra-articular resiniferatoxin on monosodium iodoacetate-induced osteoarthritic pain in rats, Korean J Physiol Pharmacol 2016; 20(1):129-136 teaches that based upon preclinical studies in rats, local delivery of Resiniferatoxin may be beneficial to osteoarthritis patients. However, the major problem with Resiniferatoxin treatment is acute toxicity. No dosages for treating humans are mentioned.

H. Aizawa et al., Int J Urol. 2016 November; 23(11):952-956. doi: 10.1111/iju.13181 relates to an examination whether systemic Resiniferatoxin treatment can desensitize the single-unit afferent activities of A- and C-fibers in rat primary bladder mechanosensitive afferent nerves. Resiniferatoxin (0.3 mg/kg) or its vehicle (10% ethanol) was injected subcutaneously to female Sprague-Dawley rats after the first eye-wipe behavior test with capsaicin.

M. J. Iadarola et al., Long-term pain relief in canine osteoarthritis by a single intra-articular injection of resiniferatoxin, a potent TRPV1 agonist, Pain. 2018 October; 159(10): 2105-2114 document a significant and prolonged analgesic effect of a single intraarticular injection of 10 μg of RTX in naturally-occurring canine osteoarthritis, which is physiologically, and symptomatically analogous to human osteoarthritis. 7 dogs with osteoarthritis were treated with a single intraarticular injection of 10 μg of Resiniferatoxin. Dose estimations, and other critical information from previous canine studies translated directly to the human cancer pain trial. This pilot study is said to provide proof-of-concept data for translation to a human osteoarthritis trial. No dosages for treating humans are quantified.

D. Leiman et al., abstract 197, Osteoarthritis and Cartilage 28 (2020) S86-S527 report about preliminary results from a phase 1b double-blind study to assess the safety, tolerability and efficacy of intraarticular Resiniferatoxin or placebo for the treatment of moderate to severe pain due to osteoarthritis of the knee. In this multicenter, randomized, double-blind, placebo-controlled study, eligible subjects aged 35-85 years, with baseline pain score in the index knee of ≥5 but 9 (WOMAC pain subscale question A1) were treated with a one-time intra-articular dose of Resiniferatoxin at escalating dose level cohorts of 5 μg, 12.5 μg, 20 μg and 30 μg in 5 or 10 mL of saline, or saline placebo. Resiniferatoxin demonstrated safety when given as onetime intraarticular injection up to the planned highest dose of 30 μg. Preliminary efficacy was also observed. Anecdotal long-term follow up suggests sustained pain relief beyond day 365.

A. Szallasi, Resiniferatoxin: Nature's Precision Medicine to Silence TRPV1-Positive Afferents, Int. J. Mol. Sci. 2023, 24, 15042 reviews medical utility of Resiniferatoxin. A breakthrough therapy designation for intraarticular Resiniferatoxin at a dose of 12.5 μg to treat pin associated with knee osteoarthritis is discussed (cf. s. Lopez et al., J. Analgesics of the Future: IA Resiniferatoxin for Osteoarthritic Knee Pain. Pract PainManag. 2022;22(2), updated Mar. 1, 2022).

US 2001 0006982 A1 (WO 1999/009970 A1) relates to a method of treating neurogenic urinary dysfunction that comprises contacting urinary bladder mucosa of a patient afflicted with neurogenic urinary dysfunction with an effective dose of a homovanilloid compound, in particular a compound selected from the group RTX, TYX, 20-homovanillyl-mezerein or 20-homovanillyl-12-deoxyphorbol-13-phenylacetate.

US 2004 0146590 A1, US 2010 0222385 A1, US 2013 0210905 A1, US 2015 0051271 A1, and US 2018 0117004 A1 provide methods and kits for the selective ablation of pain-sensing neurons. The methods comprise administration of a vanilloid receptor agonist to a ganglion in an amount that causes death of vanilloid receptor-bearing neurons.

US 2004 0161481 A1 (WO 2004/056305 A2) provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US 2005 0019258 A1 (WO 2005/004883 A2) relates to a method for the evaluation of potential therapeutic agents for the treatment of incontinence, as well as to a method for discovering new therapeutic agents for the treatment of incontinence.

US 2005 0019436 A1 (WO 2004/058286 A1) and US 2007 0036876 A1 (WO 2004/056305 A2) disclose a method for attenuating pain at a site in a human or animal in need thereof, comprising: administering at a discrete painful site in a human or animal in need thereof a single injectable or implantable dose of a capsaicinoid in an amount effective to denervate said discrete site without eliciting an effect outside the discrete location and to attenuate pain emanating from said site, said effective dose being from about 1 μg to about 5000 μg of capsaicin or a therapeutically equivalent dose of a capsaicinoid other than capsaicin.

US 2006 0269628 A1 (WO 2004/058286 A1) provides compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

WO 2008/109026 A1 relates to compositions and methods for the treatment of neuropathic or neurogenic pain and/or disorders at least partially associated with neurogenic dysfunction comprising the use of a pharmaceutical composition comprising at least one homovanilloid compound and at least one permeation enhancer.

US 2008 0139641 A1 (WO 2006/069451 A1) relates to a formulation of Resiniferatoxin for the preparation of an agent for the treatment of different pain conditions.

US 2008 0260791 A1 (WO 2004/058286 A1) relates to an injectable or implantable pharmaceutical composition for attenuating pain at a site in a human or animal in need thereof, consisting essentially of a capsaicinoid selected from the group consisting of from 1 μg to 5000 μg of capsaicin, a therapeutically equivalent amount of one or more other capsaicinoids, and combinations thereof; in a pharmaceutically acceptable vehicle for injection or implantation.

US 2009 0209633 A1 (WO 2008/011532 A2) relates to a method of treating inflammatory pain conditions that involves administering an effective amount of a TRPV1 agonist, such as Resiniferatoxin, Tinyatoxin and related potent agonists and their analogs, to a patient to selectively induce nerve terminal depolarization block and/or nerve terminal death in select TRPV1-containing neurons, to provide the desired pain relief without significant permanent damage to cell bodies of the select TRPV-1 containing neurons.

US 2010 047181 A1 (WO 2006 066419 A1) relates to a mixture comprising a vanilloid receptor agonist and a substance inhibiting nerve regeneration. Said mixture is suitable as a painkiller.

US 2010 0137344 A1 (WO 2008/015403 A1) relates to agents which are capable of inducing analgesia in chronic neuropathic pain, associated methods and uses thereof, in particular compounds capable of activating the TRPM8 receptor for the treatment of chronic neuropathic pain.

US 2014 0142073 A1 (WO 2014/075084A2) and US 2015 0133561 A1 (WO 2015/073577 A1) disclose capsaicinoid formulations and methods of treatment which can be utilized to treat/attenuate pain in mammals. Typically, administration is via injection at a discrete site to provide pain relief for an extended period of time. The formulations are administered in a pharmaceutically acceptable vehicle. The aqueous pharmaceutical compositions utilize pharmaceutically acceptable delivery vehicles that are single phase aqueous/water systems composed of pharmaceutically acceptable solvents including polyethylene glycol, ethanol and a non-ionic surfactant, e.g. polysorbate 80 (PS 80); buffers; sodium chloride and/or sugar to control osmotic pressure gradients; and hyaluronic acid to control the viscosity of the formulation and aid in formulation stability.

US 2014 0187619 A1 (WO 2013/012892 A2) relates to methods of treatment in subjects suffering from diabetes mellitus or obesity. The methods comprise the step of administering an active agent directly to the small intestine in the subject. The active agents include analgesic agents and, in particular, antinociceptive agents such as capsaicin, Resiniferatoxin, and their analogues.

US 2015 0080460 A1 relates to a method for administration of a formulation of Resiniferatoxin (RTX) directly onto the epicardium, comprising applying a formulation of RTX directly onto the epicardium under the pericardial sac.

US 2015 0190509 A1 (WO 2014/019095 A1) relates to a Resiniferatoxin solution having an enhanced storage stability, in which the Resiniferatoxin is dissolved in a body-compatible solvent which contains a protective gas in solution, wherein the amount of the protective gas is at least 1 wt.-% of the saturation amount of the protective gas in the solvent at ambient temperature and normal pressure. The solution can be used in particular for the treatment of intra-articular pain.

US 2019 0076396 A1 (WO 2019/049112 A1) relates to formulations of Resiniferatoxin (RTX) for intrathecal, intraganglionic intraarticular and pericardial administration, more specifically alcohol-free formulations of RTX comprising a solubilizing component, a monosaccharide or sugar alcohol, a saline buffer, and RTX, and having narrow ranges for pH range and specific gravity. It is alleged that for intraarticular administration, a typical volume injected into an adult knee would be from 3 ml to 10 ml, delivering a total amount of RTX from 5 ng to 50 μg, and that often the amount administered would be from 200 ng to 10 μg.

US 2019 0321493 A1 (WO 2018/094262 A2) provides materials and methods for treating regional pain. For example, compositions including one or more analgesics can be selectively administered (e.g., by image-guided injection) to one or more nerves to treat a mammal having regional pain.

WO 2020 132553A1 discloses methods of administering resiniferatoxin (RTX) perineurally for treatment of maladaptive pain, and compositions for use in such methods.

US 2020 0261409 A1 (WO 2017/087803 A1) discloses methods for treating pain using Resiniferatoxin. The Resiniferatoxin may be administered to certain anatomic sites using image-guided delivery. The Resiniferatoxin may be administered in low doses in the range of 0.5 to 3.0 μg of Resiniferatoxin in a human patient.

US 2020 0323800 A1 (WO 2015/160941 A1) relates to TRPV1 selective agonist compositions including a capsaicinoid, a surfactant and an extended release agent, and to methods of manufacture and methods of providing pain relief as well as treating a variety of disorders with such compositions.

US 2021 0007998 A1 relates to nanoparticles comprising Resiniferatoxin (RTX) encapsulated in a poly(lactic-co-glycolic acid) (PLGA) polymer and compositions, especially topical compositions, comprising the nanoparticles.

US 2021 0299090 A1 (WO 2020 139797A1) discloses a method for treating Parkinson's Disease (PD) comprising administering an effective amount of Resiniferatoxin (RTX) by an intrathecal or intracisternal administration.

US 2021 0393515 A1 relates to methods of administering Resiniferatoxin (RTX) perineurally for treatment of maladaptive pain, and compositions for use in such methods.

US 2022 0008384 A1 (WO 2020/191506 A1) relates to a composition comprising capsaicin or a capsaicinoid is used in a method for postoperative pain control. The composition is administered to a site intended for surgery in a patient at least one day before surgery is actually performed.

US 2022 0096428 A1 (WO 2020/154261 A1) relates to methods of administering Resiniferatoxin (RTX) for treatment of osteoarthritis (OA) pain, and compositions for use in such methods.

US 2023 0051321 A1 (WO 2021/209450 A1) relates to non-aqueous solid and liquid compositions comprising Resiniferatoxin and a surfactant. The non-aqueous solid and liquid compositions may be used to prepare aqueous compositions that are used in the treatment of pain, specifically osteoarthritis-related joint pain.

US 2023 0143545 A1 (WO 2021/202084 A1) relates to a method for treating pulmonary inflammatory disease comprising administering an effective amount of Resiniferatoxin (RTX) by an epidural, peri-ganglionic or intra-ganglionic administration. In some embodiments, the dose of RTX for an adult human is from about 0.1 μg to about 100 μg.

US 2023 0270713 A1 (WO 2021/257956 A1) relates to methods of administering Resiniferatoxin (RTX) intravesically for treatment of bladder pain, and compositions for use in such methods.

Further, Resiniferatoxin has been suggested for treating joint pain such as pain due to osteoarthritis of the knee. While it is alleged in US 2019 0076396 A1 (WO 2019/049112 A1) that for intraarticular administration, a typical volume injected into an adult knee would be from 3 ml to 10 ml, delivering a total amount of RTX from 5 ng to 50 μg, and that often the amount administered would be from 200 ng to 10 μg, the clinically tested doses of Resiniferatoxin in fact were in the μg scale, typically 3 μg, 6 μg, 12.5 μg, 20 μg, or 30 μg.

A completed clinical trial (www.clinicaltrials.gov, identifier NCT03542838) relates to a study of Resiniferatoxin for knee pain in moderate to severe osteoarthritis. According to the study plan, Resiniferatoxin was administered as a one-time dose, intraarticularly at a dose level of 5 μg, 12.5 μg, 20 μg, 25 μg, or 30 μg. The study was completed on Nov. 2, 2021 and no results have been posted.

A withdrawn clinical trial (www.clinicaltrials.gov, identifier NCT04044742) was planned as a phase 3 study to evaluate the efficacy and safety of Resiniferatoxin for pain due to osteoarthritis of the knee. According to the study plan, 12.5 μg of Resiniferatoxin in 5 ml volume should be administered intraarticularly as a one-time dose.

A withdrawn clinical trial (www.clinicaltrials.gov, identifier NCT04386980) was planned to evaluate resiniferatoxin in patients with knee osteoarthritis whose total knee replacement surgery is delayed. According to the study plan, 12.5 μg of Resiniferatoxin in 5 ml volume should be administered once intraarticularly.

An active, not recruiting clinical trial (www.clinicaltrials.gov, identifier NCT04885972) is planned as a study to evaluate intraarticular Resiniferatoxin to treat moderate to severe pain from knee osteoarthritis. According to the study plan, 7.5 μg, 10 μg, 12.5 μg, 15 μg, or 20 μg in 5 ml should be injected once intraarticularly.

With press release of Sep. 7, 2023, Sorrento Therapeutics announced positive phase 2a clinical trial results for Resiniferatoxin for the treatment of knee pain in moderate to severe osteoarthritis of the knee patients. The administered doses of Resiniferatoxin amounted to 7.5 to 20 μg.

The known methods and medicaments for treating knee joint pain, especially osteoarthritic knee joint pain, are not satisfactory in every respect and there is a demand for improved methods and medicaments.

Only a few pharmaceutical compositions containing Resiniferatoxin are known from the prior art.

The pharmaceutical compositions containing Resiniferatoxin that are known from the prior art are not satisfactory in every respect and there is a demand for improved pharmaceutical compositions. Especially when the administered dose of Resiniferatoxin is very low, e.g. about 400 ng, it is particularly challenging to provide satisfactory storage stability and shelf-life.

It is an object of the invention to provide pharmaceutical compositions containing Resiniferatoxin that have advantages compared to the pharmaceutical compositions of the prior art. The pharmaceutical compositions should provide satisfactory storage stability and shelf-life, should be easy to apply and administer, and should be producible on large scale in an economic manner. Further, the pharmaceutical compositions should be suitable for treating knee joint pain, especially osteoarthritic knee joint pain, should be safe and should have as little treatment emergent adverse events as possible.

This object has been achieved by the subject-matter of the patent claims.

It has been surprisingly found that pharmaceutical compositions containing comparatively low amounts of Resiniferatoxin have certain advantages when they are provided in form of solids, preferably lyophilizates. Experimental data indicate that lyophilized compositions exhibit satisfactory storage stability, not only when being stored at low temperature (e.g. −18° C. and 5° C.), but also when being stored at room temperature (25° C.). Theses advantages are particularly relevant when the amount of Resiniferatoxin per lyophilized composition is less than 3.20 μg, e.g. about 0.50 μg (500 ng).

Further, it has been surprisingly found that lyophilizates of Resiniferatoxin having low salt content or no salt content at all, especially when they contain the surfactant TPGS as main component, exhibit shortened reconstitution times, have improved robustness, avoid undesirable eutectic zones in the lyophilized cake, and can be lyophilized according to simplified freezing and evaporating protocols requiring less time and thus providing significant economic benefits. Still further, it has been surprisingly found that lyophilizates of Resiniferatoxin having low salt content have an improved cake appearance upon visual inspection, especially reduced shrinkage, and show more tendency to detach from the bottom of the vial upon reconstitution.

Still further, it has been found that especially sodium chloride has a negative impact on the quality of the cake that is obtained by lyophilization, whereas potassium chloride and calcium chloride are acceptable.

Yet further, it has been surprisingly found that reconstitution of lyophilizates of Resiniferatoxin according to the invention is significantly improved when the reconstitution liquid contains aqueous ethanol. The ethanol content in the thus obtained dose unit unexpectedly has also pharmacological advantages, especially with respect to tissue distribution being significantly enhanced.

Furthermore, it has been surprisingly found that Resiniferatoxin provides relief of knee joint pain, especially osteoarthritic knee joint pain, when it is intraarticularly administered at significantly lower doses than suggested in the prior art. While it has been suggested in the prior art to treat osteoarthritic knee joint pain by intraarticularly administering doses of Resiniferatoxin on the μg scale, e.g. 12.5 μg, it has now been surprisingly found that a much lower doses of Resiniferatoxin amounting to only 400 ng or even only 100 ng provides satisfactory analgesia, especially when the administered dose unit contains aqueous ethanol. It can be expected that such lower doses provide better tolerability and safety than the conventional doses of Resiniferatoxin on the μg scale. Moreover, it has been surprisingly found that even such ultra low doses of Resiniferatoxin provide prolonged pain relief of several months after intraarticular administration. Thus, the ultra low doses according to the invention appear particularly useful for long term treatment wherein the patient receives repeated intraarticular administration over several months or years.

### SUMMARY OF THE INVENTION

A first aspect of the invention relates to a reconstitutable solid pharmaceutical composition comprising or essentially consisting of
Resiniferatoxin in an amount of less than 3.20 μg, preferably at most 900 ng, more preferably at most 500 ng;
a surfactant; and
optionally, a buffer.

### BRIEF DESCRIPTION OF THE DRAWING

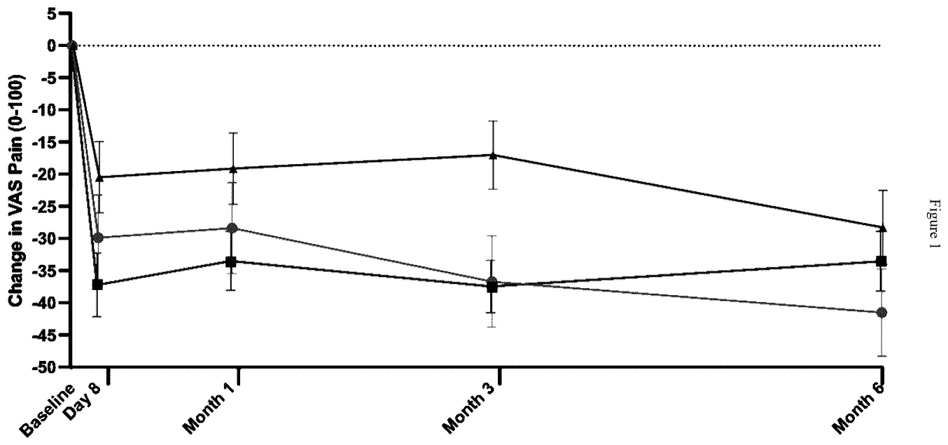

The invention will now be described in greater detail with reference to the drawing, wherein FIG. 1 is a graph that shows the efficacy in terms of visual analog scale pain in motion over time.

For the purpose of the specification, it is distinguished between
a “solid pharmaceutical composition” (also interchangeably referred to as “solid composition”);
a “reconstitution liquid”;
a “reconstituted composition”, which is obtained when the solid pharmaceutical composition and the reconstitution liquid are combined (e.g. mixed) with one another; and
a “dose unit” (also interchangeably referred to as “administered dose unit”), which may either correspond to the reconstituted composition as such, or which may be a partial volume of the reconstituted composition.

Unless expressly stated otherwise, “room temperature” means 20 to 25° C., preferably 25° C.

The solid pharmaceutical composition is for the purpose of storing low amounts of Resiniferatoxin at satisfactory storage stability and shelf-life. Preferably, every unit of solid pharmaceutical composition containing Resiniferatoxin in an amount of less than 3.20 μg, preferably at most 900 ng, more preferably at most 500 ng is for a single administration. Preferably, the solid pharmaceutical composition is contained in a container. Preferably, every container contains medication for a single administration (single dose unit).

The reconstitution liquid can be freshly prepared or stored in separate form, i.e. in a separate container. The reconstitution liquid preferably contains all those ingredients and excipients, which should be administered together with the Resiniferatoxin but which should not be contained in the solid pharmaceutical composition during storage thereof, as they might e.g. have a negative impact on storage stability and shelf-life.

The reconstituted composition is prepared shortly prior to administration, preferably by adding the reconstitution liquid to the solid pharmaceutical composition. For that purpose, the solid pharmaceutical composition is preferably contained in a container having a sufficient fill capacity such that it cannot only contain the solid pharmaceutical composition but additionally receive a predetermined volume of the reconstitution liquid. The reconstituted composition should ensure that a well defined dose of Resiniferatoxin may be taken, e.g. drawn up into a syringe, and administered as dose unit, preferably by intraarticular injection. Preferably, every dose unit of reconstituted composition containing Resiniferatoxin in an amount of less than 3.20 μg, preferably at most 900 ng, more preferably at most 500 ng is for a single administration.

Preferably, the container containing the solid pharmaceutical composition is under reduced pressure, thereby facilitating the introduction of the reconstitution liquid, and keeping a relatively low pressure inside the container. Preferably, the reduced pressure is about half atmospheric pressure. Preferably, the reduced pressure is within the range of 500±350 mbar, more preferably 500±300 mbar, still more preferably 500±250 mbar, yet more preferably 500±200 mbar, even more preferably 500±150 mbar, most preferably 500 100 mbar, and in particular 500±50 mbar.

As it will typically be difficult to completely withdraw the total volume of the reconstituted composition from the container, the volume of the reconstituted composition (and its corresponding content of Resiniferatoxin) preferably exceeds the volume of the dose unit for administration (and its corresponding administered dose of Resiniferatoxin). In other words, the volume of the administered dose unit preferably does not correspond to the total volume of the reconstituted composition that is obtained by combining the solid pharmaceutical composition and the reconstitution liquid with one another. Rather, the volume of the administered dose unit is preferably smaller than the total volume of the reconstituted composition. This facilitates withdrawal of the right dose of Resiniferatoxin from the container and subsequent administration thereof.

The solid pharmaceutical composition, the reconstituted composition, and the dose unit preferably contain medication for a single administration, preferably by injection (single dose unit). It is contemplated that the amount of Resiniferatoxin contained in the solid pharmaceutical composition and in the reconstituted composition may exceed the dose of Resiniferatoxin that is finally administered. Nonetheless, neither the solid pharmaceutical composition nor the reconstituted composition are preferably multiple dose units. On the contrary, when only a partial volume of the reconstituted composition is administered as a single dose, the excessive Resiniferatoxin remaining in the residual reconstituted composition is preferably disposed.

It is contemplated, however, that the solid pharmaceutical composition is provided for multiple treatments, i.e. after reconstitution contains more than a single dose unit for administration. For example, when the solid pharmaceutical composition is provided for treating 5 subjects, it preferably contains Resiniferatoxin in an amount of 5 times 480 ng, i.e. 2.4 μg. The reconstitution liquid preferably has a total volume of 5 times 6 ml, i.e. 30 ml. Preferably, aliquots of 5 ml thus each containing 400 ng Resiniferatoxin are drawn as dose unit into a syringe and injected into the knee of the patients.

In each case, said dose unit preferably
contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng;
contains ethanol at a volume concentration within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the dose unit; and/or
has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

Ethanol is preferably contained in an aqueous reconstitution liquid, but typically not in the solid pharmaceutical composition. The ethanol has different advantages. On the one hand, ethanol facilitates reconstitution, i.e. preparation of the reconstituted composition by mixing the solid pharmaceutical composition and the reconstitution liquid. On the other hand, ethanol improves distribution in the tissue after injection of the dose unit and thus also has a pharmacological effect.

The invention combines the advantages of administering Resiniferatoxin at very low dosages with the advantages of ethanol.

For the purpose of the specification, “essentially free” means that such component, if present at all, is present in an amount not materially affecting the essential characteristics of the mixture. Preferably, the content of such component relative to the total weight of the mixture is at most 1.0 wt.-%, more preferably at most 0.5 wt.-%, still more preferably at most 0.1 wt.-%, most preferably at most 0.001 wt.-%, and in particular below the analytical detection limit.

For the purpose of the specification, “essentially consisting of” means that specific further components can be present, namely those not materially affecting the essential characteristics of the mixture. Preferably, the total content of such further components relative to the total weight of the mixture is at most 1.0 wt.-%, more preferably at most 0.5 wt.-%, still more preferably at most 0.1 wt.-%, most preferably at most 0.001 wt.-%, and in particular below the analytical detection limit.

Preferably, the solid composition according to the invention is a lyophilizate, i.e. obtained by freeze drying (lyophilization), which is a low temperature dehydration process that involves freezing the product and lowering pressure, thereby removing the ice by sublimation. The material is cooled below its triple point ensuring that sublimation rather than melting will occur.

Lyophilizates and methods for the preparation thereof are known to the skilled person. In this regard, reference is made to e.g. L. Rey et al., Freeze Drying Lyophilization of Pharmaceutical and Biological Products, 3rd ed., informa healthcare, 2010; and D. Fissore et al., Freeze drying of pharmaceutical products, CRC Press, 2020.

The solid composition according to the invention comprises Resiniferatoxin in an amount of less than 3.20 μg, preferably at most 900 ng, more preferably at most 500 ng.

Resiniferatoxin (RTX, C₃₇H₄₀O₉, Mr 628.718 g/mol, CAS 57444-62-9, DrugBank Accession No DB06515) is the compound of the following structure:

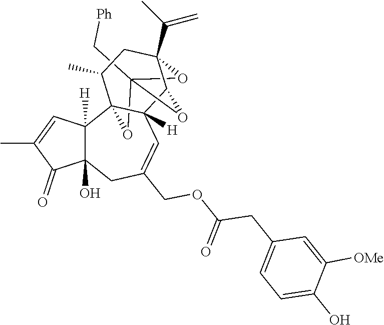 [SMILES: C=C(C)[C@]12C[C@@H](C)[C@@]34OC(Cc5ccccc5)(O[C@@H]1[C@@H]3C=C(COC(=O)Cc1ccc(O)c(OC)c1)C[C@]1(O)C(=O)C(C)=C[C@@H]41)O2]

and has the systematic name 4-hydroxy-3-methoxy-benzeneacetic acid, [(2S,3aR,3bS,6aR,9aR,9bR, 10R,11aR)-3a,3b,6,6a,9a,10,11,11a-octahydro-6a-hydroxy-8,10-dimethyl-11a-(1-methylethenyl)-7-oxo-2-(phenylmethyl)-7H-2,9b-epoxyazuleno[5,4-e]-1,3-benzodioxol-5-yl]methyl ester. It is commercially available.

Preferably, the amount of Resiniferatoxin is at least 100 ng, preferably at least 150 ng, more preferably at least 200 ng, still more preferably at least 250 ng, yet more preferably at least 300 ng, even more preferably at least 350 ng, most preferably at least 400 ng, and in particular at least 450 ng.

Preferably, the amount of Resiniferatoxin is at most 3.00 μg, preferably at most 2.75 μg, more preferably at most 2.50 μg, still more preferably at most 2.25 μg, yet more preferably at most 2.00 μg, even more preferably at most 1.75 μg, most preferably at most 1.50 μg, and in particular at most 1.25 μg.

Preferably, the amount of Resiniferatoxin is at most 1000 ng, preferably at most 900 ng, more preferably at most 800 ng, still more preferably at most 700 ng, yet more preferably at most 650 ng, even more preferably at most 600 ng, most preferably at most 550 ng, and in particular at most 500 ng.

In preferred embodiments, the amount of Resiniferatoxin is within the range of 440±200 ng, preferably 440±175 ng, more preferably 440±150 ng, still more preferably 440±125 ng, yet more preferably 440±100 ng, even more preferably 440±75 ng, most preferably 440±50 ng, and in particular 440±25 ng.

In other preferred embodiments, the amount of Resiniferatoxin is within the range of 480±200 ng, preferably 480±175 ng, more preferably 480±150 ng, still more preferably 480±125 ng, yet more preferably 480±100 ng, even more preferably 480±75 ng, most preferably 480±50 ng, and in particular 480±25 ng.

Preferably, the weight content of Resiniferatoxin is at least 3.0·10⁻⁴ wt. %, preferably at least 4.0₁₀−4 wt. %, more preferably at least 5.0·10⁻⁴ wt. %, still more preferably at least 6.0·10⁻⁴ wt. %, yet more preferably at least 7.0·10⁻⁴ wt. %, even more preferably at least 8.0·10⁻⁴ wt. %, most preferably at least 9.0·10⁻⁴ wt. %, and in particular at least 1.0·10⁻³ wt. %, relative to the total weight of the solid composition.

Preferably, the weight content of Resiniferatoxin is at most 1.0·10⁻² wt. %, preferably at most 9.0·10⁻³ wt. %, more preferably at most 8.0·10⁻³ wt. %, still more preferably at most 7.0·10⁻³ wt. %, yet more preferably at most 6.0·10⁻³ wt. %, even more preferably at most 5.0·10⁻³ wt. %, most preferably at most 4.0·10⁻³ wt. %, and in particular at most 3.0·10⁻³ wt. %, relative to the total weight of the solid composition.

Preferably, the weight content of Resiniferatoxin is within the range of 2.0(±1.8) 10⁻³ wt. %, preferably 2.0(±1.6) 10⁻³ wt. %, more preferably 2.0(±1.4) 10⁻³ wt.0%, still more preferably 2.0(±1.2) 10⁻³ wt. %, yet more preferably 2.0(±1.0) 10⁻³ wt. %, even more preferably 2.0(±0.8) 10⁻³ wt. %, most preferably 2.0(±0.6) 10⁻³ wt. %, and in particular 2.0(±0.4) 10⁻³ wt. %, relative to the total weight of the solid composition.

Preferably, the solid pharmaceutical composition according to the invention comprises a surfactant.

Preferably, the surfactant is a nonionic surfactant.

Preferably, the surfactant is a solid at room temperature.

Preferably, the surfactant is D-α-tocopherol polyethylene glycol succinate (TPGS).

Preferably, the surfactant is selected from TPGS 200, TPGS 238, TPGS 400, TPGS 600, TPGS 1000, TPGS 2000, TPGS 3400, TPGS 3500, TPGS 4000, TPGS 6000, and mixtures thereof.

Preferably, the surfactant is D-α-tocopherol polyethylene glycol 1000 succinate (TPGS 1000).

Preferably, the amount of the surfactant is at least 6.0 mg, preferably at least 8.0 mg, more preferably at least 10.0 mg, still more preferably at least 12.0 mg, yet more preferably at least 14.0 mg, even more preferably at least 16.0 mg, most preferably at least 18.0 mg, and in particular at least 20.0 mg.

Preferably, the amount of the surfactant is at most 45.0 mg, preferably at most 42.0 mg, more preferably at most 39.0 mg, still more preferably at most 36.0 mg, yet more preferably at most 33.0 mg, even more preferably at most 30.0 mg, most preferably at most 27.0 mg, and in particular at most 22.0 mg.

In preferred embodiments, the amount of the surfactant is within the range of 19.0±16.0 mg, preferably 19.0±14.0 mg, more preferably 19.0±12.0 mg, still more preferably 19.0±10.0 mg, yet more preferably 19.0±8.0 mg, even more preferably 19.0±6.0 mg, most preferably 19.0±4.0 mg, and in particular 19.0±2.0 mg

In other preferred embodiments, the amount of the surfactant is within the range of 21.0±16.0 mg, preferably 21.0±14.0 mg, more preferably 21.0±12.0 mg, still more preferably 21.0±10.0 mg, yet more preferably 21.0±8.0 mg, even more preferably 21.0±6.0 mg, most preferably 21.0±4.0 mg, and in particular 21.0±2.0 mg.

Preferably, the weight content of the surfactant is at least 45 wt. %, preferably at least 50 wt. %, more preferably at least 55 wt. %, still more preferably at least 60 wt. %, yet more preferably at least 65 wt. %, even more preferably at least 70 wt. %, most preferably at least 75 wt. %, and in particular at least 80 wt. %, relative to the total weight of the solid composition.

Preferably, the weight content of the surfactant is at most 97 wt. %, preferably at most 96 wt. %, more preferably at most 95 wt. %, still more preferably at most 94 wt. %, yet more preferably at most 93 wt. %, even more preferably at most 92 wt. %, most preferably at most 91 wt. %, and in particular at most 90 wt. %, relative to the total weight of the solid composition.

Preferably, the weight content of the surfactant is within the range of 86.5±7.0 wt. %, preferably 86.5±6.5 wt. %, more preferably 86.5±6.0 wt. %, still more preferably 86.5±5.5 wt. %, yet more preferably 86.5±5.0 wt. %, even more preferably 86.5±4.5 wt. %, most preferably 86.5±4.0 wt. %, and in particular 86.5±3.5 wt. %, relative to the total weight of the solid composition.

Preferably, the relative weight ratio of the surfactant to Resiniferatoxin is at least 5,000, preferably at least 10,000, more preferably at least 15,000, still more preferably at least 20,000, yet more preferably at least 25,000, even more preferably at least 30,000, most preferably at least 35,000, and in particular at least 40,000.

Preferably, the solid composition according to the invention comprises a buffer.

Preferably, the conjugate acid of the buffer has a pK_{A} value at 25° C. within the range of 8.1±0.8, preferably 8.1±0.7, more preferably 8.1±0.6, still more preferably 8.1±0.5, yet more preferably 8.1±0.4, even more preferably 8.1±0.3, most preferably 8.1±0.2, and in particular 8.1±0.1.

Preferably, the buffer comprises an aliphatic primary amine.

Preferably, the buffer comprises tris(hydroxymethyl)aminomethane (trometamol).

Preferably, the buffer comprises or essentially consists of a combination of tris(hydroxymethyl)aminomethane hydrochloride and tris(hydroxymethyl)aminomethane.

Preferably, the amount of the buffer is at least 0.20 mg, preferably at least 0.50 mg, more preferably at least 0.80 mg, still more preferably at least 1.10 mg, yet more preferably at least 1.40 mg, even more preferably at least 1.70 mg, most preferably at least 2.00 mg, and in particular at least 2.30 mg; preferably as equivalent weight of the conjugate base of the buffer.

Preferably, the amount of the buffer is at most 4.60 mg, preferably at most 4.30 mg, more preferably at most 4.00 mg, still more preferably at most 3.70 mg, yet more preferably at most 3.40 mg, even more preferably at most 3.10 mg, most preferably at most 2.80 mg, and in particular at most 2.50 mg; preferably as equivalent weight of the conjugate base of the buffer.

Preferably, the amount of the buffer is within the range of 2.4±1.6 mg, preferably 2.4±1.4 mg, more preferably 2.4±1.2 mg, still more preferably 2.4±1.0 mg, yet more preferably 2.4±0.8 mg, even more preferably 2.4±0.6 mg, most preferably 2.4±0.4 mg, and in particular 2.4±0.2 mg; preferably as equivalent weight of the conjugate base of the buffer.

Preferably, the weight content of the buffer is at least 2.0 wt. %, preferably at least 3.0 wt. %, more preferably at least 4.0 wt. %, still more preferably at least 5.0 wt. %, yet more preferably at least 6.0 wt. %, even more preferably at least 7.0 wt. %, most preferably at least 8.0 wt. %, and in particular at least 9.0 wt. %, relative to the total weight of the solid composition; preferably based upon the equivalent weight of the conjugate base of the buffer.

Preferably, the weight content of the buffer is at most 17 wt. %, preferably at most 16 wt. %, more preferably at most 15 wt. %, still more preferably at most 14 wt. %, yet more preferably at most 13 wt. %, even more preferably at most 12 wt. %, most preferably at most 11 wt. %, and in particular at most 90 wt. %, relative to the total weight of the solid composition; preferably based upon the equivalent weight of the conjugate base of the buffer.

Preferably, the weight content of the buffer is within the range of 10.0±4.0 wt. %, preferably 10.0±3.5 wt. %, more preferably 10.0±3.0 wt. %, still more preferably 10.0±2.5 wt. %, yet more preferably 10.0±2.0 wt. %, even more preferably 10.0±1.5 wt. %, most preferably 10.0±1.0 wt. %, and in particular 10.0±0.5 wt. %, relative to the total weight of the solid composition; preferably based upon the equivalent weight of the conjugate base of the buffer.

Preferably, the relative weight ratio of the buffer to Resiniferatoxin is at least 1,500, preferably at least 2,000, more preferably at least 2,500, still more preferably at least 3,000, yet more preferably at least 3,500, even more preferably at least 4,000, most preferably at least 4,500, and in particular at least 5,000.

Preferably, the solid composition according to the invention has a total weight of at least 5.0 mg, preferably at least 7.5 mg, more preferably at least 10 mg, still more preferably at least 12.5 mg, yet more preferably at least 15 mg, even more preferably at least 17.5 mg, most preferably at least 20.0 mg, and in particular at least 22.5 mg.

Preferably, the solid composition according to the invention has a total weight of at most 45.0 mg, preferably at most 42.5 mg, more preferably at most 40.0 mg, still more preferably at most 37.5 mg, yet more preferably at most 35.0 mg, even more preferably at most 32.5 mg, most preferably at most 30.0 mg, and in particular at most 27.5 mg.

Preferably, the solid composition according to the invention has a total weight within the range of 24.3±8.0 mg, preferably 24.3±7.0 mg, more preferably 24.3±6.0 mg, still more preferably 24.3±5.0 mg, yet more preferably 24.3±4.0 mg, even more preferably 24.3±3.0 mg, most preferably 24.3±2.0 mg, and in particular 24.3±1.0 mg.

Preferably, the solid composition according to the invention is essentially free of sodium chloride. It has been found that especially sodium chloride has a negative impact on the quality of the cake that is obtained by lyophilization, whereas potassium chloride and calcium chloride are acceptable.

Thus, while other isotonizing agents such as potassium chloride and calcium chloride may be present, sodium chloride is preferably absent. Sodium chloride is preferably contained in the reconstitution liquid, but not in the solid composition according to the invention.

Preferably, the solid composition according to the invention is essentially free of polyethylene glycol 6000 (PEG6000), preferably free of any polyethylene glycol.

Preferably, the solid composition according to the invention is essentially free of mannitol, preferably free of any bulking agent.

Preferably, the solid composition according to the invention has a residual moisture content micro-determined in accordance with Ph. Eur. 2.5.32 of at most 5.0 wt. %, preferably at most 4.5 wt. %, more preferably at most 4.0 wt. %, still more preferably at most 3.5 wt. %, yet more preferably at most 3.0 wt. %, even more preferably at most 2.5 wt. %, most preferably at most 2.0 wt. %, and in particular preferably at most 1.5 wt. %, relative to the total weight of the solid composition.

Preferably, the solid composition according to the invention has a residual moisture content micro-determined in accordance with Ph. Eur. 2.5.32 within the range of from 0.1 to 0.7 wt.-%, preferably 0.2 to 0.6 wt.-%, relative to the total weight of the solid composition.

Preferably, the solid composition according to the invention has a residual moisture content micro-determined in accordance with Ph. Eur. 2.5.32 of at most 0.32 wt. %, preferably at most 0.31 wt. %, more preferably at most 0.30 wt. %, still more preferably at most 0.29 wt. %, yet more preferably at most 0.28 wt. %, even more preferably at most 0.27 wt. %, most preferably at most 0.26 wt. %, and in particular at most 0.25 wt. %, relative to the total weight of the solid composition.

Preferably, the solid composition according to the invention has a residual moisture content micro-determined in accordance with Ph. Eur. 2.5.32 of at most 0.24 wt. %, preferably at most 0.23 wt. %, more preferably at most 0.22 wt. %, still more preferably at most 0.21 wt. %, yet more preferably at most 0.20 wt. %, even more preferably at most 0.19 wt. %, most preferably at most 0.18 wt. %, and in particular at most 0.17 wt. %, relative to the total weight of the solid composition.

Preferably, the solid composition according to the invention has a reconstitution time at 25° C. in 6.0 mL of an aqueous solution containing 4 mmol/l CaCl₂·2H₂O, 4 mmol/1 KCl, and 147 mmol/1 NaCl determined in accordance with USP of at most 50 seconds, preferably at most 45 seconds, more preferably at most 40 seconds, still more preferably at most 35 seconds, yet more preferably at most 30 seconds, even more preferably at most 25 seconds, most preferably at most 20 seconds, and in particular at most 15 seconds.

Preferably, the solid composition according to the invention has a reconstitution time at 25° C. in saline (NaCl 0.9% (w/v)) determined in accordance with USP of at most 50 seconds, preferably at most 45 seconds, more preferably at most 40 seconds, still more preferably at most 35 seconds, yet more preferably at most 30 seconds, even more preferably at most 25 seconds, most preferably at most 20 seconds, and in particular at most 15 seconds.

In preferred embodiments, the solid pharmaceutical composition according to the invention has a limited amount of isotonizing agents (isotonic agents, tonicity agents) or is essentially free of isotonizing agents.

Two preferred solid pharmaceutical compositions according to the invention can be distinguished from one another. In preferred embodiments, the solid pharmaceutical composition according to the invention comprises calcium chloride and potassium chloride. In other preferred embodiments, the solid pharmaceutical composition according to the invention is free of calcium chloride and free of potassium chloride

Preferably, the solid composition according to the invention is essentially free of potassium chloride.

Preferably, the solid composition according to the invention is essentially free of calcium chloride.

Preferably, the solid composition according to the invention is essentially free of sodium chloride, potassium chloride and calcium chloride.

Preferably, the solid composition according to the invention is essentially free of any chlorides of alkali metals and alkaline earth metals.

Preferably, the solid composition according to the invention is essentially free of any inorganic salts of alkali metals and of alkaline earth metals.

Preferably, the solid composition according to the invention is essentially free of any inorganic salts.

Preferably, the solid composition according to the invention is essentially free of any isotonizing agents.

Preferably, the solid composition according to the invention essentially consists of Resiniferatoxin, the surfactant and optionally the buffer.

In other preferred embodiments, the solid pharmaceutical composition according to the invention contains one or more isotonizing agents (tonicity agents).

Preferably, the solid composition according to the invention comprises one or more isotonizing agents.

Preferably, the one or more isotonizing agents comprise or essentially consist of inorganic salts.

Preferably, the one or more isotonizing agents comprise or essentially consist of inorganic salts of alkali metals and of alkaline earth metals.

Preferably, the one or more isotonizing agents comprise or essentially consist of chlorides of alkali metals and/or alkaline earth metals.

Preferably, the one or more isotonizing agents comprise or essentially consist of potassium chloride, calcium chloride, and a combination thereof.

Preferably, the solid composition according to the invention is essentially free of any inorganic salts other than potassium chloride and calcium chloride.

Preferably, the solid composition according to the invention essentially consists of Resiniferatoxin, the surfactant, the buffer, potassium chloride and calcium chloride.

Preferably, the total amount of the one or more isotonizing agents is at least 0.50 mg, preferably at least 0.75 mg, more preferably at least 1.00 mg, still more preferably at least 1.25 mg, yet more preferably at least 1.50 mg, even more preferably at least 1.25 mg, most preferably at least 1.50 mg, and in particular at least 1.75 mg.

Preferably, the total amount of the one or more isotonizing agents is at most 3.50 mg, preferably at most 3.25 mg, more preferably at most 3.00 mg, still more preferably at most 2.75 mg, yet more preferably at most 2.50 mg, even more preferably at most 2.25 mg, most preferably at most 2.00 mg, and in particular at most 1.80 mg.

Preferably, the total amount of the one or more isotonizing agents is within the range of 1.80±1.60 mg, preferably 1.80±1.40 mg, more preferably 1.80±1.20 mg, still more preferably 1.80±1.00 mg, yet more preferably 1.80±0.80 mg, even more preferably 1.80±0.60 mg, most preferably 1.80±0.40 mg, and in particular 1.80±0.20 mg.

Preferably, the total weight content of the one or more isotonizing agents is at least 0.5 wt. %, preferably at least 1.0 wt. %, more preferably at least 2.0 wt. %, still more preferably at least 3.0 wt. %, yet more preferably at least 4.0 wt. %, even more preferably at least 5.0 wt. %, most preferably at least 6.0 wt. %, and in particular at least 7.0 wt. %, relative to the total weight of the solid composition.

Preferably, the total weight content of the one or more isotonizing agents is at most 15.0 wt. %, preferably at most 14.0 wt. %, more preferably at most 13.0 wt. %, still more preferably at most 12.0 wt. %, yet more preferably at most 11.0 wt. %, even more preferably at most 10.0 wt. %, most preferably at most 9.0 wt. %, and in particular at most 8.0 wt. %, relative to the total weight of the solid composition.

Preferably, the total weight content of the one or more isotonizing agents is within the range of 7.0±4.0 wt. %, preferably 7.0±3.5 wt. %, more preferably 7.0±3.0 wt. %, still more preferably 7.0±2.5 wt. %, yet more preferably 7.0±2.0 wt. %, even more preferably 7.0±1.5 wt. %, most preferably 7.0±1.0 wt. %, and in particular 7.0±0.5 wt. %, relative to the total weight of the solid composition.

Preferably, the one or more isotonizing agents comprise or essentially consist of potassium chloride and wherein the amount of the potassium chloride is at least 0.20 mg, preferably at least 0.25 mg, more preferably at least 0.30 mg, still more preferably at least 0.35 mg, yet more preferably at least 0.40 mg, even more preferably at least 0.45 mg, most preferably at least 0.50 mg, and in particular at least 0.55 mg.

Preferably, the one or more isotonizing agents comprise or essentially consist of potassium chloride and wherein the amount of the potassium chloride is at most 1.40 mg, preferably at most 1.30 mg, more preferably at most 1.20 mg, still more preferably at most 1.10 mg, yet more preferably at most 1.00 mg, even more preferably at most 0.90 mg, most preferably at most 0.80 mg, and in particular at most 0.70 mg.

Preferably, the one or more isotonizing agents comprise or essentially consist of potassium chloride and wherein the amount of the potassium chloride is within the range of 0.60±0.55 mg, preferably 0.60±0.50 mg, more preferably 0.60±0.45 mg, still more preferably 0.60±0.40 mg, yet more preferably 0.60±0.35 mg, even more preferably 0.60±0.30 mg, most preferably 0.60±0.25 mg, and in particular 0.60±0.20 mg.

Preferably, the one or more isotonizing agents comprise or essentially consist of potassium chloride and wherein the content of the potassium chloride is at least 0.50 wt. %, preferably at least 0.75 wt. %, more preferably at least 1.00 wt. %, still more preferably at least 1.25 wt. %, yet more preferably at least 1.50 wt. %, even more preferably at least 1.75 wt. %, most preferably at least 2.00 wt. %, and in particular at least 2.25 wt. %, relative to the total weight of the solid composition.

Preferably, the one or more isotonizing agents comprise or essentially consist of potassium chloride and wherein the content of the potassium chloride is at most 6.00 wt. %, preferably at most 5.50 wt. %, more preferably at most 5.00 wt. %, still more preferably at most 4.50 wt. %, yet more preferably at most 4.00 wt. %, even more preferably at most 3.50 wt. %, most preferably at most 3.00 wt. %, and in particular at most 2.50 wt. %, relative to the total weight of the solid composition.

Preferably, the one or more isotonizing agents comprise or essentially consist of potassium chloride and wherein the content of the potassium chloride is within the range of 2.50±2.00 wt. %, preferably 2.50±1.75 wt. %, more preferably 2.50±1.50 wt. %, still more preferably 2.50±1.25 wt. %, yet more preferably 2.50±1.00 wt. %, even more preferably 2.50±0.75 wt. %, most preferably 2.50±0.50 wt. %, and in particular 2.50±0.25 wt. %, relative to the total weight of the solid composition.

Preferably, the one or more isotonizing agents comprise or essentially consist of calcium chloride and wherein the amount of the calcium chloride is at least 0.40 mg, preferably at least 0.50 mg, more preferably at least 0.60 mg, still more preferably at least 0.70 mg, yet more preferably at least 0.80 mg, even more preferably at least 0.90 mg, most preferably at least 1.00 mg, and in particular at least 1.10 mg.

Preferably, the one or more isotonizing agents comprise or essentially consist of calcium chloride and wherein the amount of the calcium chloride is at most 3.00 mg, preferably at most 2.75 mg, more preferably at most 2.50 mg, still more preferably at most 2.25 mg, yet more preferably at most 2.00 mg, even more preferably at most 1.75 mg, most preferably at most 1.50 mg, and in particular at most 1.25 mg.

Preferably, the one or more isotonizing agents comprise or essentially consist of calcium chloride and wherein the amount of the calcium chloride is within the range of 1.20±0.80 mg, preferably 1.20±0.70 mg, more preferably 1.20±0.60 mg, still more preferably 1.20±0.50 mg, yet more preferably 1.20±0.40 mg, even more preferably 1.20±0.30 mg, most preferably 1.20±0.20 mg, and in particular 1.20±0.10 mg.

Preferably, the one or more isotonizing agents comprise or essentially consist of calcium chloride and wherein the content of the calcium chloride is at least 1.00 wt. %, preferably at least 1.50 wt. %, more preferably at least 2.00 wt. %, still more preferably at least 2.50 wt. %, yet more preferably at least 3.00 wt. %, even more preferably at least 3.50 wt. %, most preferably at least 4.00 wt. %, and in particular at least 4.50 wt. %, relative to the total weight of the solid composition.

Preferably, the one or more isotonizing agents comprise or essentially consist of calcium chloride and wherein the content of the calcium chloride is at most 12.00 wt. %, preferably at most 11.00 wt. %, more preferably at most 10.00 wt. %, still more preferably at most 9.00 wt. %, yet more preferably at most 8.00 wt. %, even more preferably at most 7.00 wt. %, most preferably at most 6.00 wt. %, and in particular at most 5.00 wt. %, relative to the total weight of the solid composition.

Preferably, the one or more isotonizing agents comprise or essentially consist of calcium chloride and wherein the content of the calcium chloride is within the range of 4.70±4.00 wt. %, preferably 4.70±3.50 wt. %, more preferably 4.70±3.00 wt. %, still more preferably 4.70±2.50 wt. %, yet more preferably 4.70±2.00 wt. %, even more preferably 4.70±1.50 wt. %, most preferably 4.70±1.00 wt. %, and in particular 4.70±0.50 wt. %, relative to the total weight of the solid composition.

Another aspect of the invention relates to a container, preferably a glass vial, more preferably a stoppered glass vial, comprising the solid pharmaceutical composition according to the invention as described above, preferably under reduced pressure.

Preferably, the reduced pressure is within the range of 500±350 mbar, more preferably 500±300 mbar, still more preferably 500±250 mbar, yet more preferably 500±200 mbar, even more preferably 500±150 mbar, most preferably 500 100 mbar, and in particular 500±50 mbar.

Another aspect of the invention relates to the solid composition according to the invention as described above for use in the treatment of pain, preferably joint pain, more preferably knee joint pain, preferably after dilution of the solid composition with a reconstitution liquid.

Another aspect of the invention relates to a method of treating pain, preferably joint pain, more preferably knee joint pain, the method comprising the step of administering to a subject in need thereof the solid composition according to the invention as described above, preferably after dilution of the solid composition with a reconstitution liquid.

Another aspect of the invention relates to the use of Resiniferatoxin for the manufacture of the solid composition according to the invention as described above for treating pain, preferably joint pain, more preferably knee joint pain, preferably after dilution of the solid composition with a reconstitution liquid.

Preferably, the pain is associated with osteoarthritis, preferably knee osteoarthritis.

Unless expressly stated otherwise, the osteoarthritis of the knee is in accordance with the International Classification of Diseases, ICD-11 (version 1/2023), FA01.

Osteoarthritis of the knee is described therein as primary osteoarthritis occurring in an otherwise intact knee joint, involving genetically related, age-related or use-related degeneration with microscopic and macroscopic anatomical changes, which ultimately limit motion in one or more joints. Changes to the joint include increasing cartilage loss and osseous transformation such as sclerosis, osteophyte formation and cysts as well as potential inflammatory changes in surrounding soft tissue structures.

In preferred embodiments, the osteoarthritis of the knee is bilateral (XK9J), left (XK8G), right (XK9K), or unilateral unspecified (XK70).

In preferred embodiments, the pain that is associated to the osteoarthritis of the knee, i.e. the osteoarthritic knee joint pain, is selected from chronic post traumatic pain (MG30.20) and chronic secondary musculoskeletal pain associated with structural changes (MG30.31).

Within chronic post traumatic pain (MG30.20) or chronic secondary musculoskeletal pain associated with structural changes (MG30.31), the pain may have severity selected from mild pain (XS5D), moderate pain (XS9Q), and severe pain (XS2E). Within chronic post traumatic pain (MG30.20) or chronic secondary musculoskeletal pain associated with structural changes (MG30.31), the pain may have alternative severity 1 selected from mild distress (XS3R), moderate distress (XS7C), and severe distress (XS7N). Within chronic post traumatic pain (MG30.20) or chronic secondary musculoskeletal pain associated with structural changes (MG30.31), the pain may have alternative severity 2 selected from mild pain-related interference (XS5R), moderate pain-related interference (XS2L), and severe pain-related interference (XS2U). Within chronic post traumatic pain (MG30.20) or chronic secondary musculoskeletal pain associated with structural changes (MG30.31), the pain may have temporal pattern and onset selected from intermittent (XT5G), persistent (XT6Z), and persistent with overlaid attacks (XT5T).

Preferably, the pain is moderate to severe.

In preferred embodiments, the knee joint pain is osteoarthritic knee joint pain associated with osteoarthritis of Kellgren-Lawrence Grade 2 (minimal).

In preferred embodiments, the knee joint pain is osteoarthritic knee joint pain associated with osteoarthritis of Kellgren-Lawrence Grade 3 (moderate).

In preferred embodiments, the knee joint pain is osteoarthritic knee joint pain associated with osteoarthritis of Kellgren-Lawrence Grade 4 (severe).

In preferred embodiments, the knee joint pain is osteoarthritic knee joint pain associated with osteoarthritis of Ahlbäck Grade 1 (joint space<3 mm).

In preferred embodiments, the knee joint pain is osteoarthritic knee joint pain associated with osteoarthritis of Ahlbäck Grade 2 (joint space obliteration).

In preferred embodiments, the knee joint pain is osteoarthritic knee joint pain associated with osteoarthritis of Ahlbäck Grade 3 (minor bone attrition, 0-5 mm).

In preferred embodiments, the knee joint pain is osteoarthritic knee joint pain associated with osteoarthritis of Ahlbäck Grade 4 (moderate bone attrition. >5-10 mm).

In preferred embodiments, the knee joint pain is osteoarthritic knee joint pain associated with osteoarthritis of Ahlbäck Grade 5 (severe bone attrition, >10 mm).

The Ahlbäck and the Kellgren-Lawrence grading systems are known to the skilled person (S Ahlbäck, Osteoarthrosis of the knee: a radiographic investigation. Acta Radiol Suppl 1968, 277:7-72; J H Kellgren, J S Lawrence, Radiologic assessment of osteoarthritis. Ann Rheum Dis 1957 16:494-501) and can be compared to one another as follows:

| Ahlbäck | | Kellgren-Lawrence | |
|---|---|---|---|
| grade | definition | grade | definition |
| — | — | 1 | doubtful; minute osteophytes, |
| | | | doubtful significance |
| — | — | 2 | minimal; definite osteophytes, |
| | | | unimpaired joint space |
| 1 | joint space narrowing | 3 | moderate; moderate |
| | (joint space <3 mm) | | diminution of joint space |
| 2 | joint space obliteration | 4 | severe; joint space greatly of impaired |
| | | | with sclerosis subchondral bone |
| 3 | minor bone attrition (0-5 mm) | 4 | as above |
| 4 | moderate bone attrition (>5-10 mm) | 4 | as above |
| 5 | severe bone attrition (<10 mm) | 4 | as above |

Preferably, the pain is chronic.

Preferably, prior to administration the solid composition is reconstituted with a reconstitution liquid thereby obtaining a reconstituted composition. Preferably, the thus obtained reconstituted composition, either its total volume or a partial volume thereof, is devoted for subsequent administration.

In preferred embodiments, the reconstitution liquid is aqueous, preferably containing ethanol.

Preferably, the reconstitution liquid is an aqueous ethanolic solution. Preferably, the volume concentration of ethanol is within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the reconstitution liquid.

In other preferred embodiments, the reconstitution liquid is aqueous, preferably not containing ethanol.

In preferred embodiments, the reconstitution liquid is saline (NaCl 0.9% (w/v)).

Preferably, the reconstitution liquid contains a surfactant, preferably a nonionic surfactant, more preferably the same surfactant as the solid pharmaceutical composition.

Preferably, the surfactant is D-α-tocopherol polyethylene glycol succinate (TPGS).

Preferably, the surfactant is selected from TPGS 200, TPGS 238, TPGS 400, TPGS 600, TPGS 1000, TPGS 2000, TPGS 3400, TPGS 3500, TPGS 4000, TPGS 6000, and mixtures thereof.

Preferably, the surfactant is D-α-tocopherol polyethylene glycol 1000 succinate (TPGS 1000).

Preferably, the reconstitution liquid is free of mannitol, preferably free of any bulking agent.

Preferably, the reconstitution liquid is buffered.

Preferably, the reconstitution liquid has a pH value within the range of from 7.5 to 8.2.

Preferably, the reconstitution liquid has a pH value within the range of 8.0±0.8, preferably 8.0±0.7, more preferably 8.0±0.6, still more preferably 8.0±0.5, yet more preferably 8.0±0.4, even more preferably 8.0±0.3, most preferably 8.0±0.2, and in particular preferably 8.0±0.1.

Preferably, the reconstitution liquid contains one or more isotonizing agents; preferably selected from sodium chloride, potassium chloride, calcium chloride, and combinations thereof.

Preferably, the reconstitution liquid has a total volume of at least 2.5 ml, more preferably at least 3.0 ml, still more preferably at least 3.5 ml, yet more preferably at least 4.0 ml, even more preferably at least 4.5 ml, most preferably at least 5.0 ml, and in particular at least 5.5 ml.

Preferably, the reconstitution liquid has a total volume of at most 10.0 ml, preferably at most 9.5 ml, more preferably at most 9.0 ml, still more preferably at most 8.5 ml, yet more preferably at most 8.0 ml, even more preferably at most 7.5 ml, most preferably at most 7.0 ml, and in particular at most 6.5 ml.

Preferably, the reconstitution liquid has a total volume within the range of 6.0±4.0 ml, preferably 6.0±3.5 ml, more preferably 6.0±3.0 ml, still more preferably 6.0±2.5 ml, yet more preferably 6.0±2.0 ml, even more preferably 6.0±1.5 ml, most preferably 6.0±1.0 ml, and in particular 6.0±0.5 ml.

Preferably, the reconstituted composition or a partial volume thereof is administered as dose unit.

When the total volume of the reconstituted composition is administered, the administered dose corresponds to the total amount of Resiniferatoxin that is contained in the reconstituted composition and that was originally contained in the solid composition before the reconstitution liquid was added, respectively.

When a partial volume of the reconstituted composition is administered, the administered dose is smaller than the total amount of Resiniferatoxin that is contained in the reconstituted composition and that was originally contained in the solid composition before the reconstitution liquid was added, respectively.

Preferably, the administered dose unit is administered by injection, preferably by intraarticular injection, more preferably into the knee.

For intraarticular injection the patient is preferably placed in the supine position. A small pillow or towel is preferably placed under the knee thereby producing slight flexion to the hip and knee. This results in relaxation of the extensor muscles. While it is contemplated that injection may be performed via the lateral route, the medial route or the anterior route, the lateral route is preferred. After sterile preparation and draping of the skin along the lateral aspect of the patella, the patella is preferably pushed slightly laterally to open the joint. The sift tissues just inferior (posterior) to the lateral patella and its midportion can be anesthetized. For example, a 22 gauge (0.644 mm) needle may be directed anteriorly and cephalad (at about 45 degrees in each direction) until the patella is contacted. Aspiration of joint fluid is then preferably attempted.

The total volume or a partial volume of the reconstituted composition according to the invention is administered, preferably injected, into the knee joint within a few seconds or minutes. After administration is complete, the injection needle is removed.

Intraarticular administration may be monolateral or bilateral.

Preferably, the patient is a human, preferably from 35 to 85 years of age.

In preferred embodiments a patient is treated who has insufficient pain relief on optimal standard of care.

In preferred embodiments a patient is treated who is unable to be on optimal standard of care due to contraindication or intolerability.

In this regard, optimal standard of care includes one or more of non-pharmacological treatment, oral or topical treatment with NSAIDS, oral treatment with opioids, intraarticular treatment with corticosteroids, and intraarticular treatment with hyaluronic acid.

In preferred embodiments a patient is treated who prior to intraarticular administration has not been locally anesthetized at the site of intraarticular administration.

In other preferred embodiments a patient is treated who prior to intraarticular administration has been locally anesthetized at the site of intraarticular administration, preferably with Ropivacaine, Bupivacaine or Lidocaine.

Preferably, the administered dose unit has a total volume which is smaller than the total volume of the reconstituted composition.

Preferably, the administered dose unit has a total volume of at least 1.5 ml, more preferably at least 2.0 ml, still more preferably at least 2.5 ml, yet more preferably at least 3.0 ml, even more preferably at least 3.5 ml, most preferably at least 4.0 ml, and in particular at least 4.5 ml.

Preferably, the administered dose unit has a total volume of at most 9.0 ml, preferably at most 8.5 ml, more preferably at most 8.0 ml, still more preferably at most 7.5 ml, yet more preferably at most 7.0 ml, even more preferably at most 6.5 ml, most preferably at most 6.0 ml, and in particular at most 5.5 ml.

Preferably, the reconstituted composition and thus also the administered dose unit has a pH value within the range of from 7.5 to 8.2.

Preferably, the administered dose unit has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

Preferably, the administered dose unit contains Resiniferatoxin at a dose of at least 10 ng, preferably at least 25 ng, more preferably at least 50 ng, still more preferably at least 75 ng, yet more preferably at least 100 ng, even more preferably at least 125 ng, most preferably at least 150 ng, and in particular at least 175 ng.

Preferably, the administered dose unit contains Resiniferatoxin at a dose of at least 200 ng, preferably at least 225 ng, more preferably at least 250 ng, still more preferably at least 275 ng, yet more preferably at least 300 ng, even more preferably at least 325 ng, most preferably at least 350 ng, and in particular at least 375 ng.

Preferably, the administered dose unit contains Resiniferatoxin at a dose of at most 600 ng, preferably at most 575 ng, more preferably at most 550 ng, still more preferably at most 525 ng, yet more preferably at most 500 ng, even more preferably at most 475 ng, most preferably at most 450 ng, and in particular at most 425 ng.

Preferably, the administered dose unit contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng.

Preferably, the solid pharmaceutical composition is contained in a container, preferably a glass-vail, more preferably a stoppered glass vial.

In preferred embodiments, the container containing the solid pharmaceutical composition provides sufficient storage stability and shelf-life after storage at temperatures below 0° C., e.g. −18° C. Preferably, when stored at −18° C. for 6 months, the container containing the solid pharmaceutical composition contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the container prior to storage.

In other preferred embodiments, the container containing the solid pharmaceutical composition provides sufficient storage stability and shelf-life after storage at about 5° C. Preferably, when stored at 5° C. for 6 months, the container containing the solid pharmaceutical composition contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the container prior to storage.

In further preferred embodiments, the container containing the solid pharmaceutical composition provides sufficient storage stability and shelf-life after storage at room temperature (25° C.). Preferably, when stored at 25° C. for 6 months, the container containing the solid pharmaceutical composition contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the container prior to storage.

Stability is preferably determined in accordance with FDA, Guidance for Industry, Drug Stability Guidelines, and Q1A(R2) Stability Testing of New Drug Substances and Products, preferably in the version valid on Jan. 1, 2024.

Another aspect of the invention relates to a kit comprising
(i) a first kit component, which comprises a solid pharmaceutical composition according to the invention as described above; and
(ii) a second kit component, which is separate from the first kit component and comprises a reconstitution liquid according to the invention as described above.

The kit according to the invention is configured such that the second kit component may be mixed with the first kit component thereby obtaining the reconstituted composition according to the invention as described above. The reconstituted composition or a partial volume thereof may then be administered as dose unit.

Preferably, the first kit component is a container, preferably a glass vial, more preferably a stoppered glass vial, comprising the solid pharmaceutical composition under reduced pressure; preferably at a reduced pressure within the range of 500±350 mbar, more preferably 500±300 mbar, still more preferably 500±250 mbar, yet more preferably 500±200 mbar, even more preferably 500±150 mbar, most preferably 500±100 mbar, and in particular 500±50 mbar.

The kit according to the invention is preferably customized to provide upon combining the first kit component and the second kit component with one another a reconstituted aqueous ethanolic composition for use in the treatment of knee joint pain, especially osteoarthritic knee joint pain, by injection, preferably intraarticular administration, more preferably by intraarticular injection. Preferably, said reconstituted composition comprises a single unit dose of Resiniferatoxin. The kit for use according to the invention is preferably customized such that after combining the first kit component and the second kit component with one another, a predefined partial volume of the thus obtained reconstituted aqueous ethanolic composition is used as a dose unit for administration by injection. Preferably, said dose unit
contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng;
contains ethanol at a volume concentration within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the dose unit; and/or
has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

In preferred embodiments, the solid pharmaceutical composition of the first kit component is reconstituted with 6.0 ml of reconstitution liquid of the second kit component, whereas 5.0 ml of the thus reconstituted composition are subsequently administered, i.e. used in therapy.

In other preferred embodiments, the solid pharmaceutical composition of the first kit component is reconstituted with 5.5 ml of reconstitution liquid of the second kit component, whereas 5.0 ml of the thus reconstituted composition are subsequently administered, i.e. used in therapy.

Preferably, when combining the reconstitution liquid of the second kit component with the solid pharmaceutical composition of the first kit component for reconstitution, the reconstitution liquid is preferably added to the solid pharmaceutical composition by means of a syringe, preferably at a direct injection angle of 90°. Preferably, needles between 18G and 20G, preferably 20G, are used for reconstitution. Preferably, reconstitution is instituted immediately after removing the first kit component from refrigerated storage without any equilibration period at room temperature. Preferably, the same syringe is subsequently used for administration.

The solid pharmaceutical composition according to the invention is preferably a lyophilizate that may be prepared by a conventional lyophilization process. Typically, such a freeze-drying operation includes the following:
Firstly, the starting material is provided, preferably a solution of all ingredients in aqueous ethanol.
Secondly, the starting material is frozen and hardened by low temperatures (freezing step). To produce larger crystals, the product should be frozen slowly or can be cycled up and down in temperature (annealing).
Thirdly, the frozen material is placed under vacuum, is progressively heated to deliver enough energy for the ice to sublimate (sublimation phase or primary drying).
Fourthly, when the ice has been distilled away, a higher vacuum allows the progressive extraction of bound water at above-zero temperatures (desorption phase or secondary drying).
Fifthly, the product is extracted from the equipment (final conditioning and storage).
Sixthly, the product is stored under suitable conditions (ultimate storage).
Seventhly, reconstitution is achieved by adding the reconstitution liquid (reconstitution phase).

The following examples further illustrate the invention but are not to be construed as limiting its scope.

### EXAMPLE 1

Lyophilizates were prepared having the following composition:

| Ingredients | with salts | | without salts | |
|---|---|---|---|---|
| per vial | mg | [wt. %] | mg] | [wt. %] |
| Resiniferatoxin | 0.00048 | 0.00190 | 0.00048 | 0.00205 |
| TPGS | 21.00 | 83.33 | 21.00 | 89.67 |
| Tromethamine | 2.42 | 9.60 | 2.42 | 10.33 |
| CaCl₂•2H₂O | 1.18 | 4.68 | — | — |
| KCl | 0.60 | 2.38 | — | — |
| sum | 25.20 | 100.00 | 23.42 | 100.00 |

| | | |
|---|---|---|
| HCl aq. | ad pH 8 | ad pH 8 |

Water for injection and dehydrated ethanol were used as processing agents and removed during the manufacturing process.

The lyophilizate in 1 vial was reconstituted with 6 ml of a reconstitution liquid to obtain a sterile, clear, and colorless reconstituted composition for intraarticular injection. A 5 ml aliquot of the reconstituted volume, corresponding to a dose of 400 ng Resiniferatoxin, was extracted and used for intraarticular administration, while any excess volume was discarded.

### EXAMPLE 2

A lyophilized powder was prepared having the following composition:

| Ingredients per vial | mg | [wt.-%] |
|---|---|---|
| Resiniferatoxin | 0.00044 | 0.00190 |
| TPGS | 19.25 | 83.33 |
| Tromethamine | 2.22 | 9.61 |
| CaCl₂•2H₂O | 1.08 | 4.68 |
| KCl | 0.55 | 2.38 |
| sum | 23.10 | 100.00 |

| | |
|---|---|
| HCl aq. | ad pH 8 |

Water for injection and dehydrated ethanol are used as processing agents and removed during the manufacturing process.

A reconstitution liquid was provided having the following composition (total volume 6 mL):

| Ingredients per vial | mg/mL | [mg] |
|---|---|---|
| NaCl | 8.5908 | 51.54 |
| CaCl₂•2H₂O | 0.3918 | 2.35 |
| KCl | 0.1988 | 1.19 |
| Tromethamine | 0.8079 | 4.85 |
| HCl aq. | ad pH 8 | ad pH 8 |
| Dehydrated alcohol | 39.4453 | 236.67 |
| Water for injection | QS 1 mL | ad 6 mL |

The lyophilizate in 1 vial was reconstituted with 5.5 mL of the reconstitution liquid to obtain a sterile, and clear reconstituted composition for intraarticular injection. A 5 mL aliquot of the reconstituted volume, corresponding to a single dose of 400 ng Resiniferatoxin, was extracted and used for intraarticular administration, while any excess volume is discarded.

### EXAMPLE 3

The primary objective of this study was to compare the analgesic effects of single intraarticular injections of Resiniferatoxin (100 or 400 ng) compared to placebo, at 3 and 6 months postinjection, in participants with chronic osteoarthritic knee joint pain. Secondary objectives were to evaluate the analgesic effects of Resiniferatoxin versus placebo in terms of a responder analysis (percentage of subjects achieving ≥50% or ≥70% reduction in visual analog scale pain score) and changes in Western Ontario and McMaster Universities Arthritis Index (WOMAC) total and subscale scores (pain, physical function and stiffness). Safety and injection site pain were also evaluated.

The study was conducted as a randomized, double-blind, placebo-controlled, single-dose trial of Resiniferatoxin. Eligible patients were aged 40 to 80 years with radiographic knee osteoarthritis (Kellgren-Lawrence Grade 2-4 in the last 3 years) and a baseline visual analog scale (0-100 mm) pain score ≥40 mm on motion in the target knee, with or without pain medication. 67 subjects were randomized to treatment with either Resiniferatoxin 100 ng (n=24) or 400 ng (n=23), or placebo (n=20), administered intraarticularly into the index knee. Intraarticular Ropivacaine (5 ml, 0.5%) was administered 15 mins before investigational medicinal product. Visual analog scale scores were used to evaluate pain on motion as the average of the last 2 days in a target knee between baseline and at 3 and 6 months post injection. Injection site pain was rated (visual analog scale) from baseline (−0.5 h) up to 3 h post intraarticular injections.

Baseline demographic characteristics and osteoarthritis pain scores were comparable across treatment groups. In the intention-to-treat population, a reduction in the visual analog scale scores for pain on motion in the treated knee after intraarticular injection was observed as early as the first trial visit post-injection (day 8) with the effect lasting until the last trial visit (month 6; FIG. 1). Subjects receiving Resiniferatoxin 100 ng or Resiniferatoxin 400 ng reported a greater pain reduction than subjects receiving placebo. At the 3 months visit, there was a higher mean [SD] absolute reduction of visual analog scale score (baseline corrected) in the Resiniferatoxin 100 ng group (37.43 [19.79]) and the Resiniferatoxin 400 ng group (36.68 [34.16]) than in the placebo group (17.00 [23.09]). At 6 months, the mean (SD) absolute reduction of visual analog scale score was 33.52 (22.89) in the Resiniferatoxin 100 ng group, 41.48 (32.57) in the Resiniferatoxin 400 ng group, and 28.26 (25.02) in the placebo group. Consistent with these data, the Resiniferatoxin 100 ng and Resiniferatoxin 400 ng treatment groups showed a greater reduction in WOMAC total and subscale scores (pain, physical function and stiffness) than the placebo group at both 3 and 6 months.

The incidence of treatment emergent adverse events in the Resiniferatoxin 400 ng group (78.3%) and placebo group (75.0%) were comparable, but lower in the Resiniferatoxin 100 ng group (62.5%). The most commonly reported treatment emergent adverse events were arthralgia, back pain, nasopharyngitis and headache. The majority of treatment emergent adverse events were mild and unrelated to investigational medicinal product or Ropivacaine. Seven severe adverse events were reported in 6 subjects [2 subjects (Resiniferatoxin 100 ng), 3 subjects (Resiniferatoxin 400 ng) 1 subject (placebo)], none of which were considered to be related to the investigational medicinal product or Ropivacaine. No safety concerns were raised based on other evaluated safety parameters. Injection site pain (procedural pain) was highly variable across treatment groups, although generally higher following intraarticular Resiniferatoxin compared to placebo, with peak pain (moderate-severe intensity in some subjects) noted at around 0.5 h post-injection and return to baseline by 3 h.

The data from this exploratory trial indicate that single intraarticular doses of Resiniferatoxin (100 ng or 400 ng) has the potential to mediate clinically-meaningful pain relief in an osteoarthritic knee population. Analgesic onset occurred within one week of administration and was evident for at least 3 months during the follow-up period. Injection site pain was a very common event. Resiniferatoxin was found to have a good safety profile and to have been well tolerated.

FIG. 1 shows the efficacy in terms of visual analog scale pain in motion, change over time, 2-day recall, intention-to-treat population; n=number of subjects with data; VAS=visual analogue scale. Data values plotted as mean±standard error of mean, offset in X for clarity. ▴ Placebo (n=20); ▪ Resiniferatoxin 100 ng (n=24); ● Resiniferatoxin 400 ng (n=23).

### EXAMPLE 4

Reconstitution studies were conducted to investigate reconstitution process and timing for lyophilizates comprising Resiniferatoxin in accordance with Example 1.

Samples of lyophilized placebo formulations (API placebo) and samples of lyophilized Resiniferatoxin formulations according to the invention (API RTX) were reconstituted either with a conventional reconstitution liquid (in the following also referred to as “liquid saline”) or with an aqueous ethanolic buffered salt solution (in the following also referred to as “liquid diluent”). The lyophilized placebo formulations differed from the lyophilized Resiniferatoxin formulations merely in the absence of Resiniferatoxin.

The following reconstitution liquids were tested:

| | liquid saline | | liquid diluent | |
|---|---|---|---|---|
| | % (m/v) | mg/mL | % (m/v) | mg/mL |
| NaCl | 0.9 | 9 | 0.859 | 8.5908 |
| KCl | — | — | 0.020 | 0.1988 |
| CaCl₂•2H₂O | — | — | 0.039 | 0.3918 |
| concentrated HCl aq. | q.s. | q.s. | q.s. | q.s. |
| trometamol | — | — | 0.081 | 0.8079 |
| ethanol abs. | — | — | 3.945 | 39.4453 |
| water for injection | q.s. | q.s. 1 mL | q.s. | q.s. 1 mL |

The reconstitution testing was conducted incorporating both passive and active reconstitution method strategies. Passive reconstitution (mode passive) involved leaving the vial standing, while active reconstitution (mode active) involved gently swirling and inverting the vial. Vials were removed from 2-8° C. storage and subjected to reconstitution either immediately (equilibration immediately) or after leaving them at room temperature for 30 minutes (equilibration 30 minutes). Reconstitution was performed at two different injection angles, either at a 90° vertical injection angle (angle direct) or at a 450 injection angle (angle 45°). Different needles were tested, 18 gauge (needle 18G), 20 gauge (needle 20G), and 22 gauge (needle 22G). Three vials were tested under each condition (vials 1, 2 and 3) and the time (seconds) was measured until full reconstitution was achieved and a clear solution was obtained with no particulates.

The results are compiled in the following table:

| # | mode | API | liquid | needle | equilibration | angle | vial 1 | vial 2 | vial 3 | mean |
|---|---|---|---|---|---|---|---|---|---|---|
| 1a | passive | placebo | saline | 18 G | 30 minutes | 45° | 580 s | 594 s | 610 s | 594 s |
| 2a | | | | | immediately | direct | 485 s | 475 s | 490 s | 483 s |
| 3a | | | | | 30 minutes | | 435 s | 458 s | 469 s | 454 s |
| 4a | | | | 20 G | | | 538 s | 527 s | 519 s | 528 s |
| 5a | | | | 22 G | | | 567 s | 578 s | 550 s | 565 s |
| 6a | | | diluent | 20 G | immediately | | 267 s | 274 s | 281 s | 274 s |
| 7a | | RTX | | | | | 282 s | 394 s | 396 s | 397 s |
| 1b | active | placebo | saline | 18 G | 30 minutes | 45° | 295 s | 286 s | 305 s | 295 s |
| 2b | | | | | immediately | direct | 186 s | 190 s | 174 s | 183 s |
| 3b | | | | | 30 minutes | | 168 s | 175 s | 185 s | 176 s |
| 4b | | | | 20 G | | | 222 s | 194 s | 210 s | 208 s |
| 5b | | | | 22 G | | | 242 s | 234 s | 249 s | 242 s |
| 6b | | | diluent | 20 G | immediately | | 40 s | 34 s | 37 s | 37 s |
| 7b | | RTX | | | | | 54 s | 58 s | 62 s | 58 s |

As far as the method of reconstitution is concerned, it was found that passive reconstitution took longer than active reconstitution, especially when using liquid saline instead of liquid diluent. In active reconstitution, liquid saline required 10 inversions compared to 4 inversions with liquid diluent.

As far as injection angle is concerned, it was found that a direct injection angle of 900 resulted in faster reconstitution times than a 450 angle. Active reconstitution methods using a direct injection angle of 90° significantly reduced reconstitution times compared to passive methods.

As far as equilibration time is concerned, it was found that allowing freeze-dried vials to equilibrate at room temperature for 30 minutes after 2-8° C. storage had minimal impact on the reconstitution process. Initiating reconstitution immediately without equilibration resulted in quicker completion times.

As far as needle type is concerned, it was found that different needle gauges impact both passive and active reconstitution times. Narrower gauges offer more controlled and precise delivery, whereas broader gauges enable faster flow rates. For optimal results, needle sizes between 18G and 20G performed best for both active and passive reconstitution due to their wider diameters. Conversely, narrower gauges, such as those between 21G and 22G, may slightly affect the flow dynamics of the reconstitution process.

As far as training of personnel is concerned, it was found that the medical practitioner should be adequately trained in active reconstitution methods, including the importance of carrying out 10 inversions (using liquid saline) and 4 inversions (using liquid diluent). This ensures effective mixing and dissolution of the freeze-dried samples.

As far as the choice of reconstitution liquid it concerned, it was found that using liquid diluent for reconstitution significantly reduced reconstitution times, particularly in passive reconstitution, compared to liquid saline. The presence of ethanol in the liquid diluent enhanced the dissolution rate. As a result, in active reconstitution, the liquid diluent required only 4 inversions, while liquid saline required 10 inversions.

In summary, for achieving optimal results, preferably a direct injection angle of 900 is used to enhance consistency and accelerate reconstitution times. Preferably, reconstitution is instituted immediately after removing vials from refrigerated storage without any equilibration period at room temperature to improve efficiency. Preferably, 20G needles are used for reconstitution to balance reconstitution flow velocity and control. Preferably, liquid diluent is used instead of liquid saline to achieve faster reconstitution times and to improve dissolution rates.

### EXAMPLE 5

The maximum solubility of Resiniferatoxin in various liquids was investigated by the equilibrium method.

Buffer media were saturated with an excess amount of Resiniferatoxin (characterized by undissolved API) and incubated at 25° C. while shaking (150 rpm) for 72 h. After incubation, 1 mL of each sample was centrifuged (10 min, 12045 RCF) and the supernatant was used to determine the amount of solubilized Resiniferatoxin via HPLC.

The experimental results are compiled in the table here below:

| n ≥ 3, mean ± SD | Ex. 5-1 | Ex. 5-2 | Ex. 5-3 | Ex. 5-4 |
|---|---|---|---|---|
| ethanol abs. [mg/ml] | 0 | 39.44 | 39.44 | 0 |
| TPGS [mg/ml] | 3.5 | 3.5 | 3.5 | 3.5 |
| mannitol [mg/ml] | — | — | 14.9 | — |
| NaCl [mg/ml] | 8.58 | 8.58 | 8.58 | 8.58 |
| CaCl₂•2H₂O [mg/ml] | 0.2 | 0.58 | 0.58 | 0.58 |
| KCl [mg/ml] | 0.1 | 0.3 | 0.3 | 0.3 |
| trometamol [mg/ml] | 0.4 | 1.22 | 1.22 | 1.22 |
| HCl [mg/ml] | ad pH 8.0 | ad pH 8.0 | ad pH 8.0 | ad pH 8.0 |
| RTX solubility [ug/mL] | 333 ± 100 | 630 ± 38 | 336 ± 53 | 317 ± 75 |

The above experimental data demonstrate that ethanol increases maximum solubility of Resiniferatoxin, whereas mannitol decreases maximum solubility of Resiniferatoxin.

1. A reconstitutable solid pharmaceutical composition comprising or essentially consisting of
(i) Resiniferatoxin in an amount of less than 3.20 μg;
(ii) optionally, a surfactant; and
(iii) optionally, a buffer.

2. The solid composition according to claim 1, which is a lyophilizate.

3. (canceled)

4. (canceled)

5. (canceled)

6. The solid composition according to claim 1, wherein the amount of Resiniferatoxin is within the range of 440±200 ng or 480±200 ng.

7. (canceled)

8. The solid composition according to claim 1, wherein the weight content of Resiniferatoxin is at least 3.0·10⁻⁴ wt. % and at most 1.0·10⁻² wt. %, relative to a total weight of the solid composition.

9. (canceled)

10. (canceled)

11. (canceled)

12. (canceled)

13. The solid composition according to claim 1, wherein the surfactant is D-α-tocopherol polyethylene glycol succinate (TPGS) selected from TPGS 200, TPGS 238, TPGS 400, TPGS 600, TPGS 1000, TPGS 2000, TPGS 3400, TPGS 3500, TPGS 4000, TPGS 6000, and mixtures thereof.

14. (canceled)

15. (canceled)

16. (canceled)

17. (canceled)

18. (canceled)

19. The solid composition according to claim 1, wherein the weight content of the surfactant is at least 45 wt. %, relative to a total weight of the solid composition.

20. (canceled)

21. (canceled)

22. (canceled)

23. (canceled)

24. (canceled)

25. The solid composition according to claim 1, wherein the buffer comprises tris(hydroxymethyl)aminomethane (trometamol).

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. The solid composition according to claim 1, which is essentially free of polyethylene glycol 6000 (PEG6000).

39. The solid composition according to claim 1, which has a residual moisture content micro-determined in accordance with Ph. Eur. 2.5.32 of at most 5.0 wt. %, relative to a total weight of the solid composition.

40. (canceled)

41. (canceled)

42. (canceled)

43. (canceled)

44. (canceled)

45. (canceled)

46. (canceled)

47. The solid composition according to claim 1, which is essentially free of any inorganic salts.

48. (canceled)

49. (canceled)

50. The solid composition according to claim 1, which comprises one or more isotonizing agents.

51. (canceled)

52. (canceled)

53. (canceled)

54. (canceled)

55. (canceled)

56. (canceled)

57. (canceled)

58. (canceled)

59. (canceled)

60. The solid composition according to claim 50, wherein the total weight content of the one or more isotonizing agents is at least 0.5 wt. % and at most 15 wt. %, relative to a total weight of the solid composition.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. A method for treating pain in a patient in need thereof, said method comprising providing a reconstitutable solid composition according to claim 1, reconstituting the reconstitutable solid composition with a reconstitution liquid to form a reconstituted composition, and administering the reconstituted composition to the patient.

76. The method according to claim 75, wherein the pain is associated with knee osteoarthritis.

77. (canceled)

78. (canceled)

79. (canceled)

80. The method according to claim 75, wherein the reconstitution liquid is aqueous and contains ethanol.

81. (canceled)

82. The method according to claim 81, wherein the reconstitution liquid contains D-α-tocopherol polyethylene glycol succinate (TPGS) selected from TPGS 200, TPGS 238, TPGS 400, TPGS 600, TPGS 1000, TPGS 2000, TPGS 3400, TPGS 3500, TPGS 4000, TPGS 6000, and mixtures thereof.

83. (canceled)

84. (canceled)

85. (canceled)

86. (canceled)

87. (canceled)

88. The method according to claim 75, wherein the reconstitution liquid has a total volume of at most 10.0 ml.

89. (canceled)

90. The method according to claim 75, wherein the reconstituted composition or a partial volume thereof is administered as a dose unit.

91. The method according to claim 90, wherein the administered dose unit is administered by intraarticular injection.

92. (canceled)

93. (canceled)

94. (canceled)

95. (canceled)

96. The method according to claim 90, wherein the administered dose unit contains Resiniferatoxin at a dose of at least 200 ng and at most 600 ng.

97. (canceled)

98. (canceled)

99. A kit comprising
(i) a first kit component, which comprises a solid pharmaceutical composition according to claim 1; and
(ii) a second kit component, which is separate from the first kit component and comprises a reconstitution liquid.

100. (canceled)

101. (canceled)

102. (canceled)

103. (canceled)

104. (canceled)

105. (canceled)

106. (canceled)

107. (canceled)

108. The method according to claim 75, wherein the solid pharmaceutical composition is reconstituted with 6.0 ml of reconstitution liquid and 5.0 ml of the thus reconstituted composition are subsequently administered.

109. (canceled)

110. (canceled)

111. (canceled)

112. (canceled)
